SEQUENCE LISTING (3 sequences)

SEQ ID NO: 1 - protein, 28 aa, Homo sapiens
SLRRSSCFGGRMDRIGAQSGLGCNSFRY

SEQ ID NO: 2 - protein, 32 aa, Homo sapiens
SPKMVQGSGCFGRKMDRISSSSGLGCKVLRRH

SEQ ID NO: 3 - protein, 32 aa, Homo sapiens
TAPRSLRRSSCFGGRMDRIGAQSGLGCNSFRY

(19) United States
(12) Patent Application Publication
(10) Pub. No.: US 2009/0163421 A1
(43) Pub. Date: Jun. 25, 2009

(54) Room Temperature Stable, Lyophilized Natriuretic Peptide Formulations

(75) Inventors: Supriya Gupta, Sunnyvale, CA (US); Yanli Mi, San Mateo, NJ (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: EKR Therapeutics, Inc., Bedminster, NJ (US)

(21) Appl. No.: 12/340,219

(22) Filed: Dec. 19, 2008

Related U.S. Application Data
(60) Provisional application No. 61/014,986, filed on Dec. 19, 2007.

(51) Int. Cl. A61K 38/22; A61P 25/00

(52) U.S. Cl. 514/12

(57) ABSTRACT

Lyophilized pharmaceutical compositions comprise a natriuretic peptide, a buffer and a bulking agent that are stable at room temperature. Preferably, for a fill volume of 1 mL (pre-lyophilization), the total cake mass, post-lyophilization, is 30-175 mg. Preferably, the natriuretic peptide is ularitide or a pharmaceutically acceptable salt thereof. Various embodiments of the compositions may further comprise at least one of an acid and a base. In some embodiments, the pH of the compositions is between 4.0 and 6.0. In addition, various embodiments of the compositions may further comprise a stabilizing agent. In some embodiments, the compositions are isotonic. Preferably, the compositions are used for the treatment of cardiac conditions. The invention also relates to methods for preparing such compositions.

### CLAIM OF PRIORITY

This application claims priority to U.S. Provisional Application No. 61/014,986, filed Dec. 19, 2007, the disclosure of which is hereby incorporated by reference in its entirety.

### STATEMENT REGARDING SEQUENCE LISTING

Submitted herewith are sequence listings in paper and computer readable form. The information recorded in computer readable form is identical to the written sequence listing.

### BACKGROUND OF INVENTION

Natriuretic peptides are a family of peptide hormones that are synthesized by the heart, brain, endothelial cells or kidneys. Natriuretic peptides are antagonists to the renin-angiotensin-aldosterone system and thus are involved in the regulation of sodium and water balance, blood volume and arterial pressure. Natriuretic peptides exert their effects via two major pathways: vasodilatory effects and renal effects. Natriuretic peptides dilate arteries and veins, which decreases vascular resistance and pressure. Natriuretic peptides affect the kidneys by increasing glomerular filtration rate and filtration fraction, which produces natriuresis (increased sodium excretion) and diuresis (increased fluid excretion). Natriuretic peptides also affect the kidneys by decreasing the release of renin, which leads to further natriuresis and diuresis by decreasing circulating levels of angiotensin II and aldosterone. In summary, natriuretic peptides decrease blood volume, arterial pressure, venous pressure, pulmonary capillary wedge pressure and cardiac output.

The family of natriuretic peptides includes atrial natriuretic peptide (ANP), brain natriuretic peptide (BNP), C-type natriuretic peptide (CNP), dendroaspis natriuretic peptide (DNP) and urodilatin (URO or ularitide). ANP is a 28 amino acid peptide that is derived from the precursor peptide pre-pro-ANP and is synthesized in atrial myocytes. BNP is a 32 amino acid peptide that is derived from the precursor peptide pre-pro-BNP and is synthesized in the ventricles of the heart. CNP is secreted by endothelial cells. URO is a 32 amino acid peptide that is derived from the precursor peptide pre-pro-ANP and is synthesized in the kidney.

Ularitide is in clinical development for the treatment of acute decompensated heart failure. Currently, ularitide is formulated in a composition consisting of 2 mg/mL active ularitide (in acetate form) and 20 mg/mL mannitol in water. This composition is then lyophilized. The lyophilized powder is then reconstituted in physiological saline prior to administration. The current ularitide composition has several characteristics. The current ularitide formulation does not contain buffer. The lyophilized powder must also be refrigerated at 2-8° C. for maintaining product stability. Furthermore, water in the lyophilized cake accelerates product degradation. The total cake mass in the vial is low so the product is particularly susceptible to moisture-induced degradation when stored at higher temperatures.

Pharmaceutical formulations that must be refrigerated have several drawbacks. For example, the formulations must be stored at refrigerated temperatures in order to achieve a commercially viable shelf-life from a drug product supply chain perspective. Refrigeration of drug product also impacts the cost of goods. Furthermore, refrigerated products are inconvenient because they require special handling procedures prior to administration.

As a result, it would be beneficial to develop a stable, room temperature natriuretic peptide formulation that does not require refrigeration during storage and still has a commercially viable shelf-life. These benefits may be enhanced if the formulation is packaged in an ad-mix container, where the drug product vial is attached to an IV bag, so that the reconstitution steps are minimized, which also reduces errors in dose preparation and offers convenience. Furthermore, such a formulation will be advantageous in an emergency room setting where it is critical to have drug product presentations that minimize handling and preparation steps.

The pharmaceutical compositions of the present invention meet the each of above needs.

### BRIEF SUMMARY OF THE INVENTION

The invention relates to lyophilized pharmaceutical compositions comprising a natriuretic peptide, a buffer and a bulking agent that are stable at room temperature. Preferably, for a fill volume of 1 mL (pre-lyophilization), the total cake mass, post-lyophilization, is 30-175 mg. Preferably, the natriuretic peptide is ularitide or a pharmaceutically acceptable salt thereof. Various embodiments of the compositions may further comprise at least one of an acid and a base for pH adjustment. In some embodiments, the pH of the compositions is between 4.0 and 6.0. In addition, various embodiments of the compositions may further comprise a stabilizing agent. In some embodiments, the compositions are isotonic. Preferably, the compositions are used for the treatment of cardiac conditions. The invention also relates to methods for preparing such compositions.

In certain embodiments, the present invention is directed to a lyophilized natriuretic peptide composition having a moisture content of less than about 1.0% w/w when stored for 3 months at 40° C. and 75% RH.

In certain embodiments, the present invention is directed to a lyophilized natriuretic peptide composition having a moisture content of at least about 0.1% w/w or at least about 0.5% w/w when stored for 3 months at 40° C. and 75% RH.

In certain embodiments, the present invention is directed to a lyophilized natriuretic peptide composition having a moisture content of less than about 2.0% w/w, less than about 1.5% w/w, or less than about 1.0% w/w when stored for 6 months at 40° C. and 75% RH.

In certain embodiments, the present invention is directed to a lyophilized natriuretic peptide composition having a moisture content of at least about 0.1% w/w or at least about 0.5% w/w when stored for 6 months at 40° C. and 75% RH.

In certain embodiments, the present invention is directed to a lyophilized natriuretic peptide composition having a moisture content of less than about 2.5% w/w, less than about 2.0% w/w, less than about 1.5% w/w, or less than about 1.0% w/w when stored for 9 months at 40° C. and 75% RH.

In certain embodiments, the present invention is directed to a lyophilized natriuretic peptide composition having a moisture content of at least about 0.1% w/w or at least about 0.5% w/w when stored for 9 months at 40° C. and 75% RH.

In certain embodiments, the present invention is directed to a lyophilized natriuretic peptide composition having a moisture content of less than about 3.0% w/w less than about 2.5% w/w, less than about 2.0% w/w, less than about 1.5% w/w, or less than about 1.0% w/w when stored for 12 months at 40° C. and 75% RH.

In certain embodiments, the present invention is directed to a lyophilized natriuretic peptide composition having a moisture content of at least about 0.1% w/w or at least about 0.5% w/w when stored for 12 months at 40° C. and 75% RH.

In certain embodiments, the present invention is directed to a lyophilized natriuretic peptide composition which maintains a purity of the natriuretic peptide of at least about 90%, at least about 93%, at least about 95%, at least about 97% or at least about 99% when stored for 9 months at 40° C. and 75% RH.

In certain embodiments, the present invention is directed to a lyophilized natriuretic peptide composition which maintains a purity of the natriuretic peptide of at least about 90%, at least about 93%, at least about 95%, at least about 97% or at least about 99% when stored for 12 months at 40° C. and 75% RH.

In certain embodiments, the present invention is directed to a lyophilized natriuretic peptide composition which maintains a purity of the natriuretic peptide of at least about 90%, at least about 93%, at least about 95%, at least about 97% or at least about 99% when stored at room temperature for 12 months.

In certain embodiments, the present invention is directed to a lyophilized natriuretic peptide composition which maintains a purity of the natriuretic peptide of at least about 90%, at least about 93%, at least about 95%, at least about 97% or at least about 99% when stored at room temperature for 18 months.

In certain embodiments, the present invention is directed to a lyophilized natriuretic peptide composition which maintains a purity of the natriuretic peptide of at least about 90%, at least about 93%, at least about 95%, at least about 97% or at least about 99% when stored at room temperature for 24 months.

In certain embodiments, the present invention is directed to a lyophilized natriuretic peptide composition which maintains a pH with about 10%, within about 5% or within about 3% when stored at room temperature for 12 months.

In certain embodiments, the present invention is directed to a lyophilized natriuretic peptide composition which maintains a pH with about 10%, within about 5% or within about 3% when stored at room temperature for 18 months.

In certain embodiments, the present invention is directed to a lyophilized natriuretic peptide composition which maintains a pH with about 10%, within about 5% or within about 3% when stored at room temperature for 24 months.

In certain embodiments, the present invention is directed to a lyophilized natriuretic peptide composition which maintains osmolality with about 10%, within about 5% or within about 3% when stored at room temperature for 12 months.

In certain embodiments, the present invention is directed to a lyophilized natriuretic peptide composition which maintains osmolality with about 10%, within about 5% or within about 3% when stored at room temperature for 18 months.

In certain embodiments, the present invention is directed to a lyophilized natriuretic peptide composition which maintains osmolality with about 10%, within about 5% or within about 3% when stored at room temperature for 24 months.

In certain embodiments, the present invention is directed to a lyophilized natriuretic peptide composition which maintains a purity of the natriuretic peptide of at least about 95%, at least about 97%, at least about 99% when stored for 4 weeks at 60° C.

In certain embodiments, the present invention is directed to a lyophilized natriuretic peptide composition which maintains a purity of the natriuretic peptide of at least about 96%, at least about 98%, at least about 99% when stored for 9 weeks at 60° C.

In certain embodiments, the present invention is directed to a lyophilized natriuretic peptide composition which maintains a purity of the natriuretic peptide of at least 99%, at least 99.3%, at least about 99.7% when stored for 8 weeks at 45° C.

In certain embodiments, the present invention is directed to a lyophilized natriuretic peptide composition which maintains a purity of the natriuretic peptide of at least about 99%, at least about 99.5%, at least about 99.9% when stored for 13 weeks at 45° C.

In certain embodiments, the present invention is directed to a lyophilized natriuretic peptide composition which maintains a purity of the natriuretic peptide of at least 99%, at least 99.3%, at least about 99.6% when stored for 6 weeks at 45° C.

In certain embodiments, the present invention is directed to a lyophilized natriuretic peptide composition which maintains a purity of the natriuretic peptide of at least about 98.5%, at least about 99.3%, at least about 99.8% when stored for 8 weeks at 45° C.

In certain embodiments, the present invention is directed to a lyophilized natriuretic peptide composition which maintains a purity of the natriuretic peptide of at least 98%, at least 99%, at least about 99.5% when stored for 16 weeks at 45° C.

In certain embodiments, the present invention is directed to a lyophilized natriuretic peptide composition which maintains a purity of the natriuretic peptide of at least about 96%, at least about 98%, at least about 99% when stored for 29 weeks at 45° C.

In certain embodiments, the present invention is directed to a method of treating a acute decompensated heart failure comprising administering a composition as disclosed herein to a patient in need thereof.

In certain embodiments, the present invention is directed to a method of treating a acute decompensated heart failure comprising administering a reconstituted composition as disclosed herein to patient in need thereof.

In certain embodiments, the present invention is directed to a method of preparing a pharmaceutical composition comprising:(a) forming a liquid formulation comprising natriuretic peptide, a bulking agent, and a buffer; (b) decreasing the temperature of the formulation over at least 10 minutes to a temperature between 0° C. and 10° C., maintaining the temperature for at least 10 minutes and decreasing the temperature over at least 10 minutes to below −30° C.; (c)annealing the formulation to a point between −15° C. and 0° C. and maintaining the temperature for at least 90 minutes; (d) decreasing the temperature over at least 10 minutes to below −30° C. and maintaining the temperature for at least 60 minutes; and (e) drying the formulation.

In certain embodiments, the present invention is directed to a method of preparing a pharmaceutical composition comprising: (a) forming a liquid formulation comprising natriuretic peptide, a bulking agent, and a buffer; (b) decreasing the temperature of the formulation over at least 10 minutes to a temperature between 0° C. and 10 degrees C.; maintaining the temperature for at least 10 minutes; reducing the temperature over at least 10 minutes to a temperature between −15° C. and 0° C.; maintaining the temperature for at least 10 minutes; and decreasing the temperature over at least 10 minutes to below −30° C.; (c) annealing the formulation to a point between −15° C. and 0° C. and maintaining the temperature for at least 90 minutes; (d) decreasing the temperature over at least 10 minutes to below −30° C. and maintaining the temperature for at least 60minutes; and (e) drying the formulation.

In certain embodiments, the drying step comprises a primary drying step to a temperature below 0° C., maintaining the temperature for at least 10 minutes followed by a secondary drying step to a temperature above 0° C.

In other embodiments, the drying step comprises a primary drying step carried out at −30° C. to −10° C. at a pressure of 100 to 200 mTorr for a duration of 30-40 hours followed by a secondary drying step carried out at 25° C. to 35° C. at a pressure of 50-150 mTorr for a duration of 6-10 hours.

### BRIEF DESCRIPTION OF THE DRAWINGS

FIGS. 1-4 are a series of graphs illustrating the stability of three current ularitide drug product (Current DP) lots in terms of both water content and peptide purity over time.

FIG. 5 is a graph comparing the product purity over time for several formulations of the invention to the current ularitide drug product after reconstitution.

### DETAILED DESCRIPTION OF THE INVENTION

### Definitions

The term “natriuretic peptide”, as used herein, means a peptide that has the biological activity of promoting natriuresis, diuresis or vasodilation. Assays for testing such activity are known in the art, e.g., as described in U.S. Pat. Nos. 4,751,284 and 5,449,751. Examples of natriuretic peptides include, but are not limited to, atrial natriuretic peptide (ANP or ANP(99-126)), brain natriuretic peptide (BNP), C-type natriuretic peptide (CNP), dendroaspis natriuretic peptide (DNP), urodilatin (URO or ularitide), and any fragments of the prohormone ANP(1-126) or BNP precursor polypeptide that retain vasodilating, natriuretic or diuretic activity. For further examples of natriuretic peptides and their use or preparation, see, e.g., U.S. Pat. Nos. 4,751,284; 4,782,044; 4,895,932; 5,449,751; 5,461,142; 5,571,789; and 5,767,239. See also, Ha et al., Regul. Pept. 133(1-3):13-19, 2006. The term natriuretic peptide also broadly encompasses a peptide having an amino acid sequence substantially identical, for example, having a sequence identity at least 80% or 85%, more preferably at least 90%, 95%, or even higher, to a naturally occurring natriuretic peptide (e.g., ANP or URO). Typically, such a peptide may include one, two, three, four, five or more amino acids that have been modified from the naturally occurring sequence by addition, deletion or substitution. Furthermore, the term natriuretic peptide encompasses any peptide having the amino acid sequence of a naturally occurring natriuretic peptide with chemical modification, e.g., deamidation, phosphorylation, PEGylation, etc., at one or more residues, or substitution by the corresponding D-isomer(s), so long as the peptide retains a portion, e.g., at least 1%, preferably 10%, more preferably 50%, and most preferably at least 80%, 90% or higher, of the biological activity of the corresponding wild-type natriuretic peptide.

The terms “urodilatin” and “ularitide” generally refer to a 32-amino acid peptide hormone that is described in U.S. Pat. No. 5,449,751 and has the amino acid sequence set forth in GenBank Accession No. 1506430A. Uroditatin, the 95-126 fragment of atrial natriuretic peptide (ANP), is also referred to as ANP(95-126). The terms “atrial natriuretic peptide” or “ANP(99-126)” or “ANP” refer to a 28-amino acid peptide hormone that is transcribed from the same gene and derived from the same polypeptide precursor, ANP(1-126), as urodilatin, but without the first four amino acids at the N-terminus. For a detailed description of the prohormone, see, e.g., Oikawa et al. (Nature 1984; 309:724-726), Nakayama et al. (Nature 1984; 310:699-701), Greenberg et al. (Nature 1984; 312:656-658), Seidman et al. (Hypertension 1985; 7:31-34) and GenBank Accession Nos. 1007205A, 1009248A, 1101403A, and AAA35529. The polynucleotide sequence encoding this prohormone is provided in GenBank Accession No. NM 6172.1. Conventionally, the term “urodilatin” (URO) is used to refer to the naturally occurring peptide, whereas the term “ularitide” is used to refer to the recombinantly produced or chemically synthesized peptide. In this application, the terms “urodilatin” and “ularitide” are used interchangeably to broadly encompass both a naturally occurring peptide and a recombinant or synthetic peptide. The terms “urodilatin” and “ularitide” also encompass any peptide of the above-cited amino acid sequence containing chemical modification (e.g., deamidation, phosphorylation, PEGylation, etc.) at one or more residues or substitution by the corresponding D-isomer(s), so long as the peptide retains biological activity as a natriuretic peptide. Furthermore, a chemically modified urodilatin or ularitide may contain one or more amino acid substitutions for the purpose of facilitating the desired chemical modification (e.g., to provide a reactive group for conjugation). “Urodilatin” or “ularitide”, regardless of whether it contains chemical modifications or amino acid sequence modification, retains a portion, i.e., at least 1%, preferably 10%, more preferably 50%, and most preferably at least 80% or 90%, of the biological activity of the naturally-occurring wild-type urodilatin or ANP(95-126).

The terms “pharmaceutical formulation” and “pharmaceutical composition”, which are used herein interchangeably, refer to a composition comprising an active pharmaceutical ingredient in combination with one or more pharmaceutically acceptable excipients or carriers. The pharmaceutical formulations of the present invention allow the peptides or salts thereof to remain physically, chemically and biologically stable.

The term “stable” or “stability”, as used in the context of the present invention, means that the peptide composition retains its physical stability and/or chemical stability and/or biological activity upon storage. Various analytical techniques for measuring peptide stability for predetermined times and temperatures are well known in the art and are reviewed in, e.g., “Peptide and Protein Drug Delivery,” 247-301, Vincent Lee Ed., Marcel Dekker, Inc., New York, N.Y., Pubs(1991), and Jones, A. Adv. Drug Delivery Rev. 10:29-90(1993). Stability may be measured, for example, after exposure to a selected temperature for a selected time period. A peptide retains its physical stability in a pharmaceutical formulation if it shows no significant decrease in purity and potency upon RP-HPLC, or no significant changes in color and/or clarity upon visual examination and/or UV spectroscopy.

A “stable” liquid formulation or a “stable” lyophilized formulation is a liquid formulation or lyophilized formulation comprising a peptide or salt thereof that exhibits no significant physical, chemical, or biological changes in the peptide when stored at a refrigerated temperature (2-8° C.) for at least 12 months, preferably 2 years, and more preferably 3 years; or at room temperature (22-28° C.) for at least 1 year, and preferably, 18-24 months. The criteria for stability for the current ularitide product is as follows: no more than 10%, and preferably no more than 5%, of peptide is degraded as measured by RP-HPLC (drug purity); the potency (drug concentration) of the drug should remain within 90-110% relative to the concentration claimed on the label; preferably, the reconstituted solution remains colorless, or clear to slightly opalescent by visual analysis; and the concentration, pH and osmolality of the formulation have no more than ±10% change.

The term “pharmaceutically acceptable”, as used herein, means compatible with the other ingredients of the formulation, and not deleterious to the recipient thereof. Similarly, the term “pharmaceutically acceptable salt” means salt forms of the active compounds that are prepared with counter ions that are non-toxic under the conditions of use and are compatible with a stable formulation.

The terms “lyophilization”, “lyophilize” and “freeze-dried” is a freeze-drying process that is often used in the preparation of pharmaceutical compositions to preserve their biological activity. The process generally involves a pre-lyophilized liquid form or starting solution that is subsequently frozen, sublimating the previously frozen liquid sample in a vacuum in order to remove the ice and/or other frozen solvent, and thereby leaving the non-solvent components intact in the form of a powdery or cake-like substance. During the lyophilization process, an excipient may be included in the pre-lyophilized liquid formulation to enhance the stability of the lyophilized product upon storage. The lyophilized product can be stored for prolonged periods of time and at elevated temperatures without loss of biological activity, and can readily be reconstituted into a particle-free solution by the addition of an appropriate diluent. An appropriate diluent can be any physiologically acceptable liquid in which the lyophilized powder is completely soluble. Sterile pyrogen-free water or sterile pyrogen-free saline are preferred diluents. An advantage of lyophilization is that the water content is reduced to a level that greatly reduces the various water related molecular events that can lead to instability of the peptides and proteins upon long-term storage.

The term “lyoprotectant” includes agents that provide stability to the protein during the lyophilization process (primary and secondary drying cycles), presumably by providing an amorphous glassy matrix and by binding with the protein or protein derivative through hydrogen bonding and replacing the water molecules that are removed during the drying process. This helps maintain the protein conformation, minimize protein degradation during the lyophilization cycle and improve the long-term stability of the protein or protein derivative. Lyoprotectants are well known in the art and commercially available. Examples include, but are not limited to, polyols or sugars, such as sucrose and trehalose.

The term “cryoprotectant” generally includes agents that stabilize the protein or protein derivative against freezing-induced stresses during the lyophilization process. They may also offer protection during primary and secondary drying, and long-term product storage. Cryoprotectants are well known in the art and commercially available. Examples of cryoprotectants include, but are not limited to, polymers, such as dextran and polyethylene glycol; sugars, such as sucrose, glucose, trehalose, and lactose; surfactants, such as polysorbates; and amino acids, such as glycine, arginine, and serine.

The term “reconstitution time”, as used herein, is the time that is required to rehydrate a lyophilized formulation with a pharmaceutically acceptable liquid to form a particle-free clarified solution.

The term “room temperature”, as used herein, means a temperature between 65-75° F. or 22-28° C.

The term “patient”, as used herein, means human and non-human animals.

### Description

The invention relates to lyophilized pharmaceutical compositions comprising a natriuretic peptide, a buffer and a bulking agent that are stable at room temperature. Preferably, for a fill volume of 1 mL (pre-lyophilization), the total cake mass, post-lyophilization, is 30-175 mg. The invention also relates to methods for making such compositions.

The pharmaceutical compositions of the present invention comprise a natriuretic peptide or a pharmaceutically acceptable salt thereof. However, the compositions may comprise multiple natriuretic peptides. Examples of natriuretic peptides include, but are not limited to, atrial natriuretic peptide (ANP or ANP(99-126)), brain natriuretic peptide (BNP), C-type natriuretic peptide (CNP), dendroaspis natriuretic peptide (DNP), urodilatin (URO or ularitide), or fragments or modified versions thereof. Examples of pharmaceutically acceptable salts of natriuretic peptides, such as ularitide, include, but are not limited to, acetates, hydrochlorides, sulfates, phosphates, acetates, fumarates, maleates and tartarates. A preferred natriuretic peptide is ularitide. A preferred pharmaceutically acceptable salt of ularitide is ularitide acetate.

Natriuretic peptides are well known in the art and commercially available. Preferably, ANP is a 28 amino acid peptide that has the amino acid sequence of SEQ ID NO: 1. Preferably, BNP is a 32 amino acid peptide that has the amino acid sequence of SEQ ID NO: 2. Preferably, CNP is a 22 amino acid peptide. Preferably, URO is a 32 amino acid peptide that has the amino acid sequence of SEQ ID NO: 3.

In some embodiments, the pharmaceutical compositions comprise 0.1-25 mg/mL of a natriuretic peptide. For example, suitable concentrations of natriuretic peptide, include, but are not limited to: 0.1-1 mg/mL, 1-5 mg/mL, 5-10 mg/mL, 10-15 mg/mL, 15-20 mg/mL, 20-25 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL and 25 mg/mL. Preferably, the compositions comprise 1-5 mg/mL ularitide or pharmaceutically acceptable salt thereof. Most preferably, the compositions comprise 2.0 mg/mL ularitide (in acetate form).

The pharmaceutical compositions also comprise a buffer. However, the compositions may comprise multiple buffers. In general, the compositions should have sufficient buffering capacity to maintain the pH of the solution in an acceptable range throughout the shelf-life of the product. In some embodiments, the buffer concentration is 1-25 mM. For example, suitable buffer concentrations include, but are not limited to: 1-5 mM, 5-10 MM, 10-15 mM, 15-20 mM, 20-25 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM or 25 mM. Preferably, the buffer concentration is 5-10 mM. Most preferably, the buffer concentration is 10 mM. Buffers are well known in the art and commercially available. Examples of typical buffers include, but are not limited to, glutamate, citrate, tartrate, benzoate, lactate, histidine or other amino acids, gluconate, phosphate, malate, succinate, formate and propionate. Preferably, the buffer is succinate or histidine. More preferably, the buffer is histidine.

In some embodiments, the pH of the compositions is between 3.0 and 7.0. For example, suitable pH ranges include, but are not limited to: 3.0-4.0, 4.0-5.0, 5.0-6.0 or 6.0-7.0. In further embodiments, the pH of the compositions is between 4.0 and 5.5. In other embodiments, the pH of the compositions is from 4.0 to 5.0. In additional embodiments, the pH of the compositions is from 5.5 to 6.0. The most preferred pH range is 4.0-6.0. The pH of the pharmaceutical compositions affects both the solubility and the stability of the natriuretic peptides in the compositions. Generally, the drug substance degrades faster in solution as the pH of the composition increases.

In various embodiments, the pharmaceutical compositions may, optionally, further comprise at least one of an acid and a base in order to adjust the pH of the compositions. As discussed above, if the buffer alone does not achieve the desired pH for the composition, then the pH of the composition may be adjusted with an acid or a base. Acids and bases are well known in the art and commercially available. Examples of typical acids include, but are not limited to, hydrochloric acid, phosphoric acid, citric acid, ascorbic acid, acetic acid, sulphuric acid, carbonic acid and nitric acid. Examples of typical bases include, but are not limited to, sodium hydroxide, potassium hydroxide, calcium hydroxide, sodium carbonate and magnesium hydroxide. Acids and bases may be added on an as needed basis in order to achieve a desired pH. A preferred acid is hydrochloric acid. A preferred base is sodium hydroxide.

The pharmaceutical compositions also comprise a bulking agent. However, the compositions may comprise multiple bulking agents. Bulking agents are used to increase the total mass of the lyophilized powder cake, to reduce residual moisture levels in the cake, and provide additional structure to the freeze-dried product. In addition to providing a pharmaceutically acceptable cake, bulking agents may also impart useful qualities to the lyophilized compositions, such as modifying the collapse temperature in order to alter the lyophilization process conditions, providing freeze-thaw protection, and further enhancing the protein stability over long-term storage. These agents may also serve as tonicity modifiers. In some embodiments, the bulking agent concentration is 1-10% (10 mg/mL-100 mg/mL). For example, suitable concentrations of bulking agent include, but are not limited to: 1-3% (10 mg/mL-30 mg/mL), 3-8% (30 mg/mL-80 mg/mL), 8-10% (80 mg/mL-100 mg/mL), 1% (10 mg/mL), 2% (20 mg/mL), 3% (30 mg/mL), 4% (40 mg/mL), 5% (50 mg/niL), 6% (60 mg/mL), 7% (70 mg/mL), 8% (80 mg/mL), 9% (90 mg/mL), 10% (100 mg/mL). Preferably, for a fill volume of 1 mL (pre-lyophilization), the total cake mass, post-lyophilization, is 30-175 mg. More preferably, for a fill volume of 1 mL (pre-lyophilization), the total cake mass, post-lyophilization, is 40-110 mg. Most preferably, for a fill volume of 1 mL (pre-lyophilization), the total cake mass, post-lyophilization, is 50-100 mg. Bulking agents are well known in the art and commercially available. Examples of typical bulking agents include, but are not limited to, mannitol, glycine, lactose and sucrose.

In various embodiments, the pharmaceutical compositions may, optionally, further comprise a stabilizing agent. However, the compositions may comprise multiple stabilizing agents. Stabilizing agents, such as cryoprotectants and lyoprotectants, are used to stabilize the pharmaceutical compositions. These agents may also serve as tonicity modifiers. In some embodiments, the stabilizing agent concentration is 1-10% (10 mg/mL-100 mg/mL). For example, suitable concentrations of stabilizing agent include, but are not limited to: 1-3% (10 mg/mL-30 mg/mL), 3-6% (30 mg/mL-60 mg/mL), 6-10% (60 mg/mL-100 mg/mL), 1% (10 mg/mL), 2% (20 mg/mL), 3% (30 mg/mL), 4% (40 mg/mL), 5% (50 mg/mL), 6% (60 mg/mL), 7% (70 mg/mL), 8% (80 mg/mL), 9% (90 mg/mL) or 10% (100 mg/mL). The preferred range is 1-3% (10 mg/ml-30 mg/ml). Preferably, for a fill volume of 1 mL (pre-lyophilization), the total cake mass, post-lyophilization, is 30-175 mg. More preferably, for a fill volume of 1 mL (pre-lyophilization), the total cake mass, post-lyophilization, is 40-110 mg. Most preferably, for a fill volume of 1 mL (pre-lyophilization), the total cake mass, post-lyophilization, is 50-100 mg. Stabilizing agents are well known in the art and commercially available. Examples of typical stabilizing agents include, but are not limited to, sucrose and trehalose, mannitol and glycine.

The pharmaceutical compositions of the present invention are lyophilized. The following criteria are important for developing stable lyophilized protein or protein derivative containing formulations: protein unfolding during lyophilization should be minimized; the glass transition temperature (Tg) of the lyophilized powder should be greater than the product storage temperature; residual moisture should be low (<1-2% w/w); a preferred shelf-life is at least 1 year, and, 18-24 months at room temperature; the reconstitution time should be short, for example, less than 5 minutes, preferably less than 2 minutes, and more preferably less than 1 minute; when the lyophilized product is reconstituted, the reconstituted sample should be stable for at least 24-48 hours at 2-8° C.

The pharmaceutical compositions of the present invention are first made as a pre-lyophilized liquid formulation. A short term (e.g., 24 hour) liquid formulation stability is important to help prevent purity or potency loss at 25° C. prior to lyophilization. The pre-lyophilized formulation is then lyophilized to form a dry, stable powder that can be easily reconstituted to a particle-free solution suitable for administering to humans. Preferably, samples are kept frozen for ˜10 hours at —40° C. before initiating the primary drying cycle. A preferred freezing includes an annealing step at —10° C. to —15° C. for ˜4-5 hours before the primary drying. A preferred primary drying cycle is carried out from −30° C. to −10° C. at a pressure of 150 mTorr for the duration of 30-40 hours. A preferred secondary drying cycle is carried out at 25° C. and 35° C. at a pressure of 50-150 mTorr for a duration of 6-10 hours.

In various embodiments, the lyophilized pharmaceutical compositions are packaged in a pharmaceutically acceptable container. As used herein, the term “pharmaceutically acceptable container” means a container closure system that: protects the drug product, for example, from factors that can cause degradation of the dosage form over its shelf-life; is compatible with the drug product, for example, the packaging components will not interact sufficiently to cause unacceptable changes in the quality of either the drug or the packaging component, such as absorption or adsorption of the drug substance, degradation of the drug substance that is induced by extractables/leachables from the container, precipitation, and changes in pH; and is safe, for example, a container that does not leach harmful or undesirable amounts of substances to which a patient will be exposed when being treated with the product. Pharmaceutically acceptable containers include, but are not limited to, intravenous bags, bottles, vials and syringes. However, the surface that comes into direct contact with the drug solution has the most direct impact on product stability. Preferably, the pharmaceutically acceptable container is a Type 1 glass vial or syringe with a stopper that is suitable for a lyophilized drug product.

The pharmaceutical compositions are used to treat cardiac conditions. Preferably, the compositions are used to treat conditions that are alleviated by the administration of natriuretic peptides, such as heart failure.

The following examples disclose specific embodiments of the pharmaceutical compositions of the present invention that are illustrative of the invention as a whole and are not to be construed as limiting the scope of the invention.

### EXAMPLES

Stability data regarding the current ularitide drug product are shown in Example 1. Specific embodiments of pharmaceutical compositions of the invention are shown in Example 2. Examples 3-5 illustrate experiments and results using specific embodiments of the invention.

### Example 1

### Stability of the Current Ularitide Drug Product

The current ularitide drug product is a lyophilized formulation consisting of 2 mg/mL ularitide and 20 mg/mL mannitol in water. The lyophilized powder is refrigerated between 2-8° C. and then reconstituted in physiological saline prior to administration. This formulation was lyophilized using the cycle parameters for manufacturing the current clinical drug product (labeled as current DP in Table 4).

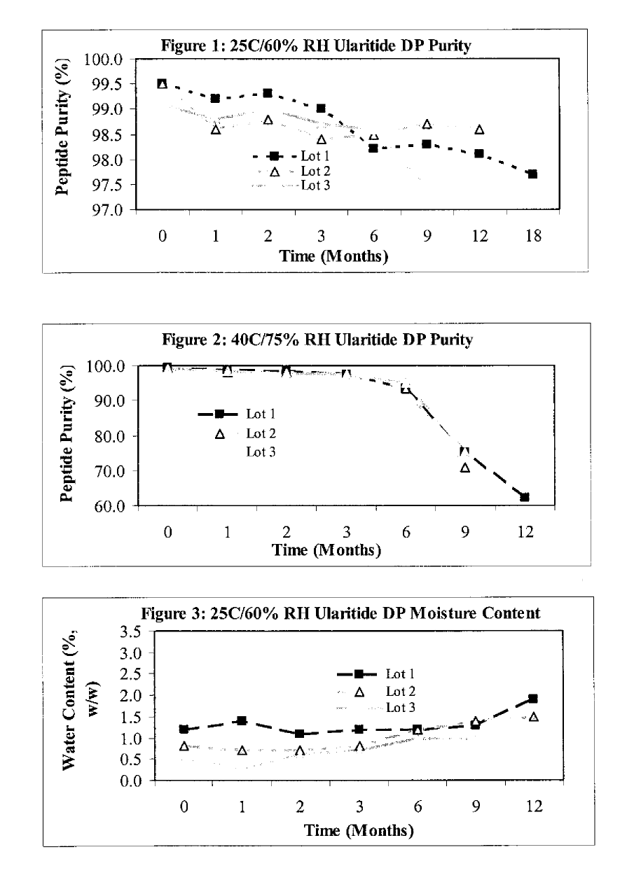

In this study, the water content (%, w/w) and peptide purity (%) of three lots of the current ularitide drug product were monitored as a function of time under two different storage conditions. Under one condition, the temperature was maintained at 25° C. with 60% relative humidity (RH). Under another condition, the temperature was maintained at 40° C. with 75% RH. Samples stored under these conditions were evaluated for stability by measuring both the water content and the peptide purity. The percentage of water content was determined by the Karl-Fisher method. The percentage of peptide purity was determined by RP-HPLC. The results of these studies are shown in FIGS. 1-4.

As shown in FIGS. 1-4, the moisture content increased over time and the peptide purity decreased over time for products stored at both conditions. For all three lots, there was a significant decrease in product purity from 95-100% to less than 70-80% observed at 40° C. when moisture levels approached 3% w/w at the 9 month time point. This data suggests that the product degrades rapidly as the moisture levels approach 3% during storage. The increase in moisture over time is typically observed for lyophilized drug product because of moisture transfer from the stoppers that undergo an autoclave and drying cycle prior to use. The presented 12-month results indicate the formulation is potentially susceptible to moisture-induced degradation at higher temperatures. The new formulations have improved compositions such that moisture induced degradation is minimized and a commercially viable, robust, room temperature stable drug product is achieved.

### Example 2

### Formulation Preparation and Analysis

The formulations were compounded, filtered, filled, and lyophilized using aseptic techniques. During compounding, appropriate buffers, bulking agents, stabilizing agents and ularitide were added in a step-wise manner. First, the buffers were prepared in water for injection (WFI); the pH was adjusted, if needed, by adding an acid or base; bulking agent and/or stabilizing agent were added, if needed; active ularitide was added to the above solution right before the filtering, filling and lyophilization process; and sufficient water or buffer was added to reach the final target volume. The lyophilization cycle parameters used for preparing the drug product are shown in Table 4.

The following pharmaceutical compositions listed in Tables 1, 2 and 3 were prepared according to the above procedures. The stability results for the pharmaceutical compositions described in Tables 1, 2, and 3 are described later in Examples 3-5.

TABLE 1
Composition of Formulations Described in Example 3

| Formula | | Active | Bulking | |
|---|---|---|---|---|
| ID | Buffer | Ingredient | Agent | Stabilizer |
| 1 | 10 mM | 2 mg/mL | 4% Mannitol | Not added |
| 2 | Succinate, | Ularitide¹ | | 1% Sucrose |
| 3 | pH 4.0 | | | 1% Trehalose |
| 4 | | | | 1% Glycine |
| 5 | 10 mM | | 4% Glycine | Not added |
| 6 | Succinate, | | | 1% Sucrose |
| 7 | pH 4.0 | | | 1% Trehalose |
| 8 | | | | 1% Mannitol |
| 9 | 10 mM | | 4% Mannitol | Not added |
| 10 | Histidine, | | | 1% Sucrose |
| 11 | pH 5.5 | | | 1% Trehalose |
| 12 | | | | 1% Glycine |
| 13 | 10 mM | | 4% Glycine | Not added |
| 14 | Histidine, | | | 1% Sucrose |
| 15 | pH 5.5 | | | 1% Trehalose |
| 16 | | | | 1% Mannitol |
| 17 | Water | | 2% Mannitol | Not added |
| (Control 1)² | | | | |
| Current DP | Water | | 2% Mannitol | Not added |
| (Control 2)³ | | | | |

¹Ularitide acetate is the active ingredient salt form.
²Formulation 17 (control 1) has the same composition as the Current DP but was lyophilized using the new cycle conditions in Table 4 for the new formulations.
³Current DP (control 2) has the same composition as formulation 17, but was lyophilized using the cycle parameters in Table 4 for the current clinical drug product (labeled as current DP).

TABLE 2
Composition of Formulations Described in Example 4

| | | Active | Bulking | |
|---|---|---|---|---|
| Formula ID | Buffer | Ingredient | Agent | Stabilizer |
| 18 | 10 mM Succinate, | 2 mg/mL | 5% Mannitol | 1% Sucrose |
| 19 | pH 4.0 | Ularitide¹ | 6% Glycine | |
| 20 | 10 mM Histidine, | | 5% Mannitol | Not added |
| 21 | pH 5.5 | | | 1% Sucrose |
| 22 | | | 5% Glycine | Not added |
| 23 | | | | 1% Sucrose |
| 17 | Water | | 2% Mannitol | Not added |
| (Control 1)² | | | | |
| Current DP | Water | | 2% Mannitol | Not added |
| (Control 2)³ | | | | |

¹Ularitide acetate is the active ingredient salt form.
²Formulation 17 (control 1) has the same composition as the Current DP but was lyophilized using the new cycle conditions in Table 4.
³Current DP (control 2) has the same composition as formulation 17, but was lyophilized using the cycle parameters in Table 4.

TABLE 3
Composition of Formulations Described in Example 5

| | | Active | Bulking | |
|---|---|---|---|---|
| Formula ID | Buffer | Ingredient | Agent | Stabilizer |
| 24 | 10 mM Histidine, | 2 mg/mL | 3% Glycine | Not added |
| 25 | pH 5.5 | Ularitide¹ | 8% Glycine | Not added |
| 26 | | | 3% Glycine | 2% Sucrose |
| 27 | | | 8% Glycine | 2% Sucrose |
| 28 | | | 5.5% | 1% Sucrose |
| | | | Glycine | |
| Current DP | Water | | 2% Mannitol | Not added |
| (Control 2)³ | | | | |

¹Ularitide acetate is the active ingredient salt form.
³Current DP (control 2) has the same composition as formulation 17, but was lyophilized using the cycle parameters in Table 4).

TABLE 4
Key Lyophilization Cycle Parameters Used to Prepare the Samples for
Examples 3, 4 and 5

| | Current Drug Product | New Cycle Parameters for New |
|---|---|---|
| Lyophilization | Cycle Parameters (Current | Formulations (Examples 3, 4 and |
| Steps | DP) | 5) |
| Freezing 1 | +5° C. to −45° C. in 100 min | 25° C. to +5° C. in 40 min; hold at 5° C. |
| | and holds at −45° C. for 120 min | for 20 min |
| | | +5° C. to −10° C. in 30 min; hold at −10° C. |
| | | for 30 min |
| | | −10° C. to −40° C. in 30 min; hold at −40° C. |
| | | for 60 min |
| Annealing | 45° C. to −15° C. in 60 min; | −40° C. to −10° C. in 60 min; hold at −10° C. |
| | hold at −15° C. for 60 min | for 240 min |
| Freezing 2 | −15° C. to −45° C. in 60 min; | −10° C. to −40° C. in 30 min; hold at −40° C. |
| | hold at −45° C. for 60 min | for 90 min |
| Primary drying | −45° C. to −5° C. in 80 min; hold | −40° C. to −30° C. in 40 min; hold at −30° C. |
| | at −5° C. for 480 min; | 30° C. for 1920-2400 min; Vacuum: |
| | Vacuum: 300 microns | 150 microns |
| Secondary drying | −5° C. to +35° C. in 80 min; | −30° C. to 25° C.-35° C. in 220-260 min; |
| | hold at +35° C. for 480 min; | hold at 25° C. for 360-480 min; |
| | +35° C. to +5° C. in 60 min; | 25° C. to 40° C. in 60 min; hold at |
| | hold at +5° C. for 60 min; | 40° C. for 120 min; (optional); |
| | Vacuum: 300 microns | Vacuum: 50 microns |

For example, in Table 1, Formula 1 contains 10 mM succinate buffer at pH 4.0, 2 mg/ml ularitide and 4% mannitol; Formula 2 contains 10 mM succinate buffer at pH 4.0, 2 mg/ml ularitide, 4% mannitol and 1% sucrose; Formula 3 contains 10 mM succinate buffer at pH 4.0, 2 mg/ml ularitide, 4% mannitol and 1% trehalose; and Formula 4 contains 10 mM succinate buffer at pH 4.0, 2 mg/ml ularitide, 4% mannitol and 1% glycine.

### Example 3

### Initial Stability Screening of Ularitide Compositions as a Function of pH and Buffer Stored at 60° C.

The stability of 2 mg/mL ularitide compositions was evaluated as a function of pH in various buffer systems. The compositions studied were pharmaceutical compositions listed in Table 1 of Example 2. The lyophilization cycle parameters for preparing the new formulations, as well as the current DP, are described in Table 4. The study controls included the formulation 17 (control 1) with 2% w/v mannitol in water for injection (WFI). Formulation 17 has the same composition as the Current DP, but was lyophilized using the new cycle conditions in Table 4 for other new formulation. Another study control (control 2) is the current DP, the same composition as formulation 17, but was lyophilized using the lyophilization cycle conditions in Table 4 for manufacturing the current clinical drug product.

Each of the compositions was prepared according to the method in Example 2. Stability was monitored by RP-HPLC at the initial time point, T=0 (post lyophilization) and following storage at 60° C. The percentage drop in product purity from the initial, T=O, value after storage for up to 9 weeks at 60° C. is reported in Table 5. Some formulations were not measured for stability after 4 weeks due to the significant drop of the purity. The study results indicate that the drug product is not stable at pH 4.0 in succinate buffer for formulations containing mannitol or glycine. A significant decrease in product purity (>15%) was observed for these formulations relative to the control formulations after 4 weeks storage. The mannitol and glycine formulations at pH 5.5 demonstrated improved stability relative to the pH 4.0 succinate formulations. In addition, the formulations containing glycine, with or without the sugars sucrose or trehalose, showed the most promising stability, as the decrease in purity for these formulations was lower than the current DP and formulation 17 (both controls). Both controls have the same composition, but were lyophilized using different cycle parameters described in Table 4.

TABLE 5
RP-HPLC Results: Drop in Product Purity for Formulation Candidates after 9
Weeks Storage at 60° C.

| | | | % Drop in Purity from | |
|---|---|---|---|---|
| | | | T = 0 (DP Stored at 60° C.) | |
| Formula ID | Formulation Description | pH | T = 4 wks | T = 9 wks |
| 1 | Mannitol_Succinate pH4 | 4.0 | 69.4 | NM |
| 2 | Mannitol_Sucrose_SuccinatepH4 | | 72.0 | NM |
| 3 | Mannitol_Trehalose_SuccinatepH4 | | 46.7 | NM |
| 4 | Mannitol_Glycine_SuccinatepH4 | | 66.7 | NM |
| 5 | Glycine_SuccinatepH4 | 4.0 | 30.7 | NM |
| 6 | Glycine_Sucrose SuccinatepH4 | | 16.0 | NM |
| 7 | Glycine_Trehalose_SuccinatepH4 | | 18.5 | NM |
| 8 | Glycine_Maninitol_SuccinatepH4 | | 26.8 | NM |
| 9 | Mannitol_HistidinepH5.5 | 5.5 | 2.8 | 4.4 |
| 10 | Mannitol_Sucrose_HistidinepH5.5 | | 7.0 | 11.6 |
| 11 | Mannitol_Trehalose_HistidinepH5.5 | | 5.0 | 6.2 |
| 12 | Mannitol_Glycine_Histidine_pH5.5 | | 22.9 | 26.6 |
| 13 | Glycine_Histidine_pH5.5 | 5.5 | 1.4 | −0.1 |
| 14 | GlycineSucrose HistidinepH5.5 | | 1.3 | 1.7 |
| 15 | Glycine_Trehalose_HistidinepH5.5 | | 1.2 | −0.3 |
| 16 | Glycine_Mannitol Histidine_pH5.5 | | 2.9 | 3.3 |
| 17 (Control | Mannitol_20 mg/mL WFI | NM | 3.4 | 2.3 |
| 1) | | | | |
| Current DP | Mannitol_20 mg/mLLWFI | | NM | 4.9 |
| (Control 2) | | | | |

NM: Not measured.

The results from this study helped identify promising formulation compositions. However, because the product storage temperature was significantly higher than the intended storage temperature of 25° C., a follow-on study was done at a lower storage temperature of 45° C. in order to confirm the trends observed in this study.

### Example 4

### Stability of Ularitide Compositions as a Function of pH and Buffer Stored at 45° C.

The stability of 2 mg/mL ularitide compositions was evaluated as a function of pH in various buffer systems. The compositions studied were pharmaceutical compositions 17-23 in Table 2 of Example 2. The buffers evaluated were 10 mM succinate at pH 4.0 and 10 mM histidine at pH 5.5. The bulking agents evaluated in the compositions were mannitol at a concentration of 5% w/v (50 mg/mL) and glycine at a concentration of 6% w/v (60 mg/mL). The concentrations of the bulking agents in this example were increased from the previous example based upon the visual cake appearance findings of the previous study in Example 3, which demonstrated that a higher total cake mass resulted in more cohesive and elegant cakes. The lyoprotectant (stabilizing agent) sucrose was evaluated in this study at a concentration of 1% w/v (10 mg/mL).

The compositions studied were pharmaceutical compositions listed in Table 2 of Example 2. The lyophilization cycle parameters for preparing the new formulations, as well as the current DP, are described in Table 4. The study controls included the formulation 17 (control 1) with 2% w/v mannitol in water for injection (WFI). Formulation 17 has the same composition as the Current DP, but was lyophilized using the new cycle conditions in Table 4 for other new formulation. Another study control (control 2) is the current DP, the same composition as formulation 17, but was lyophilized using the lyophilization cycle conditions in Table 4 for manufacturing the current clinical drug product.

Each of the compositions was prepared according to the method in Example 2 and monitored on stability by pH, W, moisture content and RP-HPLC. The percentage drop in product purity from the initial, T=0, value after storage for up to 13 weeks at 45° C. is reported in Table 6. As observed in the previous study in Example 3, the study results indicate that the product stability is not favorable at pH 4.0 in 10 mM succinate buffer for formulations containing mannitol or glycine. Furthermore, the pH 5.5 formulations with both mannitol and glycine demonstrated improved stability profiles relative to the control formulations. At the lower storage temperature of 45° C., the formulations at pH 5.5 showed a slightly lower decrease in product purity levels relative to the control manntiol formulations prepared with either cycle. Stability results for pH, moisture content and UV concentration are not shown. Briefly, moisture levels were lower than 1.0% for all formulations. Potency trends were consistent with the purity changes presented in Table 6 and pH changes were minimal for all evaluated formulations.

TABLE 6
RP-HPLC Results: Drop in Product Purity for Formulation Candidates after
8 Weeks and 13 Weeks Storage at 45° C.

| | | | % Drop in Purity from | |
|---|---|---|---|---|
| | | | T = 0 (DP Stored at 45° C.) | |
| Formula ID | Formulation Description | pH | T = 8 wks | T = 13 wks |
| 18 | Mannitol_Sucrose_Succinate_pH4 | 4.0 | 10.5 | 21.3 |
| 19 | Glycine_Sucrose_Succinate_pH4 | | 2.5 | 4.7 |
| 20 | Mannitol_Histidine_pH5.5 | 5.5 | 0.9 | 0.7 |
| 21 | Mannitol_Sucrose_Histidine pH5.5 | | 0.9 | 0.5 |
| 22 | Glycine_Histidine_pH5.5 | | 0.4 | 0.1 |
| 23 | Glycine_Sucrose_Histidine_pH5.5 | | 0.3 | −0.2 |
| 17 (Control 1) | Mannitol_20 mg/mL_WFI | NM | 2.1 | 2.0 |
| Current DP | Mannitol_20 mg/mL_WFI | | 1.2 | 2.3 |
| (Control 2) | | | | |

NM: Not measured.

Based upon these study results, histidine buffer of 10 mm at pH 5.5 was selected for further evaluation. Since these preliminary results were promising with the bulking agents mannitol and glycine, further evaluations were continued with both excipients.

The impact of sugars on product stability was not clear from these preliminary screening stability studies. Therefore, further studies continue to evaluate the stability of formulations with and without the sugar sucrose.

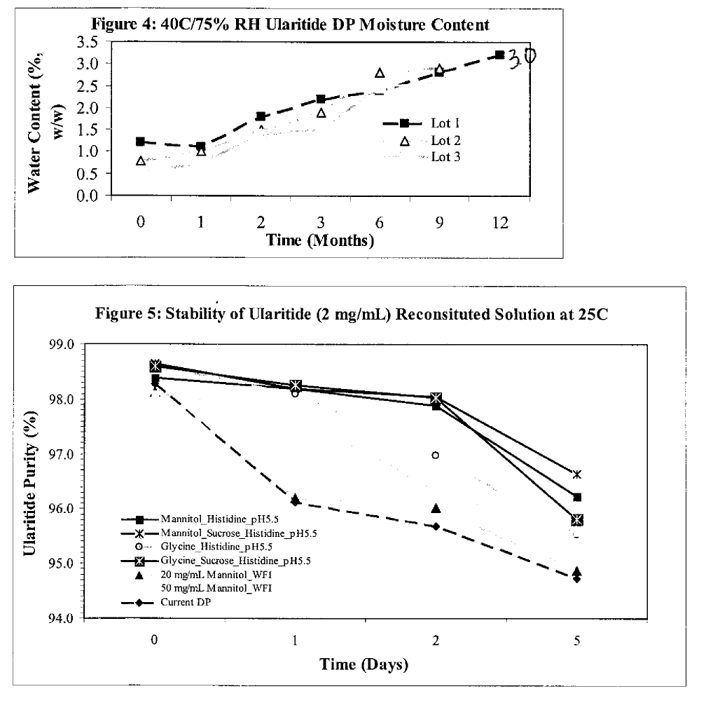

Because the data from all previous studies suggest that the formulations in the pH 5.5 histidine buffer are more stable, the solution stability of some formulations at pH 5.5, in 10 mM histidine buffer was evaluated for 5 days at 25° C. after reconstitution with WFI. Evaluating the stability of the product in solution is important because it provides an assessment of potential drug degradation during the processing of the lyophilized drug product, as well as when the product is reconstituted to a solution prior to administration. Adequate solution stability for at least 24 hours is important for meeting the processing requirements for this drug product during manufacture, and minimizing degradation during the intended infusion in the clinic. Based upon the RP-HPLC results in FIG. 5, the stability of all formulations in pH 5.5 histidine buffer demonstrated a 0.2% to 0.5% decrease in product purity after 24 hours. In comparison, the current DP formulations showed greater than 2.0% purity decrease after 1 day of reconstitution. Therefore, results show that the stability of the drug product in solution is improved for the histidine formulations relative to the current formulation.

### Example 5

Stability of Ularitide in Histidine Buffer at pH 5.5 with Different Combinations of Glycine and Sucrose

The stability of 2 mg/mL ularitide was evaluated in 10 mM histidine buffer at pH 5.5 with various concentrations of bulking agents and stabilizing agents. The compositions studied are listed in Table 3 of Example 2. This study evaluated the bulking agent glycine in the concentration range of 3-8% w/v. The effect of the sugar sucrose was also evaluated in the concentration range of 0-2% w/v.

The study controls included the current drug formulation with mannitol in WFI. This formulation was lyophilized using the cycle used currently for manufacturing the current clinical drug product (labeled as current DP). Each of the compositions was prepared according to the method in Example 2. The percentage drop in product purity from the initial, T=0, value after storage for up to 29 weeks at 45° C. is reported in Table 7. pH changes were not observed. The potency trend changes were consistent with the purity changes presented in Table 7.

TABLE 7
RP-HPLC Results: Drop in Product Purity for Glycine-Sucrose Formulations
after 29 Weeks Storage at 45° C..

| | | | % Drop in Purity from T = 0 (DP Stored | | | |
|---|---|---|---|---|---|---|
| | Formulation | | at 45° C.) | | | |
| Formula ID | Description | pH | T = 6 wks | T = 8 wks | T = 16 wks | T = 29 wks |
| 24 | 3% Glycine_0% | 5.5 | 1.3 | 0.8 | 1.4 | 1.4 |
| | Sucrose_pH5.5 | | | | | |
| 25 | 8% Glycine_0% | | 1.6 | 1.0 | 1.3 | 1.6 |
| | Sucrose_pH5.5 | | | | | |
| 26 | 3% Glycine_2% | | 0.4 | 0.2 | 0.5 | 0.4 |
| | Sucrose_pH5.5 | | | | | |
| 27 | 8% Glycine_2% | | 0.9 | 0.7 | 0.8 | 0.4 |
| | Sucrose_pH5.5 | | | | | |
| 28 | 5.5% Glycine_1% | | 1.2 | 0.6 | 0.7 | 0.6 |
| | Sucrose_pH5.5 | | | | | |
| Current DP | Mannitol 20 | NM | 1.4 | 1.9 | 2.7 | 4.3 |
| (Control 2) | mg/mL_WFI | | | | | |

NM: Not measured. Formulation does not contain a buffer and pH was not controlled.

As observed in the previous studies, the study results indicate that the pH 5.5 formulations with glycine in the concentration range of 3-8% have an improved stability profile relative to the control formulations. Furthermore, the combination of glycine with sucrose in the formulations appears to improve product stability. For example, the formulations with 3-8% glycine and 1-2% sucrose show −0.4-0.6% drop in product purity relative to the T=0 measurement after product storage at 45° C. for over 6 months. These data support the fact that the new pharmaceutical compositions provide conditions that would enable the development of a robust room temperature stable drug product.

Glycine formulations showed significant improvement on HPLC stability. However, because promising results were also observed with the higher concentration mannitol formulations, two formulations with mannitol were also selected for long-term stability evaluation. Based upon these study results, the following formulations, pharmaceutical formulations 20, 21, 22, and 23 listed in Table 2 were selected for long-term stability evaluation at 5, 25 and 40° C. relative to the current DP formulation:
2 mg/mL ularitide, 10 mM histidine formulation, pH 5.5, 5% w/v mannitol (Formulation #20);
2 mg/mL ularitide, 10 mM histidine formulation, pH 5.5, 5% w/v mannitol, 1% sucrose. (Formulation #21);
2 mg/mL ularitide, 10 mM histidine formulation, pH 5.5, 6% w/v glycine; (Formulation #22); and
2 mg/mL ularitide, 10 mM histidine formulation, pH 5.5, 6% w/v glycine, 1% sucrose (Formulation #23).

Long-term stability evaluation for up to 24 months is currently ongoing for these lead formulation candidates (pharmaceutical formulations 20-23) at 5, 25 and 40° C. relative to the current DP formulation.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

We claim:

1. A lyophilized pharmaceutical composition comprising:
(a) a natriuretic peptide;
(b) a bulking agent; and
(c) a buffer.

2. The composition of claim 1 wherein the natriuretic peptide is selected from the group consisting of atrial natriuretic peptide, brain natriuretic peptide, C-type natriuretic peptide, dendroaspis natriuretic peptide and ularitide.

3. The composition of claim 2 wherein the natriuretic peptide is ularitide.

4. The composition of claim 1 wherein the bulking agent is selected from the group consisting of mannitol and glycine.

5. The composition of claim 1 wherein the buffer is histidine.

6. The composition of claim 1 wherein the composition further comprises a stabilizing agent.

7. The composition of claim 6 wherein the stabilizing agent is selected from the group consisting of sucrose and trehalose.

8. The composition of claim 1 wherein the composition further comprises at least one of an acid and a base.

9. The composition of claim 8 wherein the acid is hydrochloric acid.

10. The composition of claim 8 wherein the base is sodium hydroxide.

11. The composition of claim 1 wherein the pH of the composition is 4.0-6.0.

12. The composition of claim 1 wherein the lyophilized composition has a post-lyophilization total cake mass of 30-175 mg for a fill volume of 1 mL (pre-lyophilization).

13. The composition of claim 1 comprising:
(a) 0.1-10 mg/mL natriuretic peptide;
(b) 1-10% bulking agent; and
(c) 1-25 mM buffer.

14. The composition of claim 1 comprising:
(a) 0.1-10 mg/mL ularitide;
(b) 1-10% bulking agent; and
(c) 1-25mM buffer.

15. The composition of claim 1 comprising:
(a) 0.1-10 mg/mL ularitide;
(b) 1-10% bulking agent; and
(c) 1-25 mM buffer;
wherein the pH of the composition is 4.0-6.0.

16. The composition of claim 1 comprising:
(a) 0.1-10 mg/mL ularitide;
(b) 1-10% bulking agent;
(c) 1-25 mM buffer; and
(d) 1-10% stabilizing agent.

17. The composition of claim 1 comprising:
(a) 0.1-10 mg/mL ularitide;
(b) 1-10% bulking agent;
(c) 1-25 mM buffer; and
(d) 1-10% stabilizing agent;
wherein the pH of the composition is 4.0-6.0.

18. The composition of claim 15 comprising:
(a) 2 mg/mL ularitide;
(b) 5% mannitol; and
(c) 10 mM histidine;
wherein the pH of the composition is 5.5.

19. The composition of claim 17 comprising:
(a) 2 mg/mL ularitide;
(b) 5% mannitol;
(c) 10 mM histidine; and
(d) 1% sucrose;
wherein the pH of the composition is 5.5.

20. The composition of claim 15 comprising:
(a) 2 mg/mL ularitide;
(b) 6% glycine; and
(c) 10 mM histidine;
wherein the pH of the composition is 5.5.

21. The composition of claim 17 comprising:
(a) 2 mg/mL ularitide;
(b) 6% glycine;
(c) 10 mM histidine; and
(d) 1% sucrose,
wherein the pH of the composition is 5.5.

22. The composition of claim 15 comprising:
(a) 1-3 mg/mL ularitide;
(b) 3-7% mannitol; and
(c) 8-12 mM histidine;
wherein the pH of the composition is 5.0-6.0.

23. The composition of claim 17 comprising:
(a) 1-3 mg/mL ularitide;
(b) 3-7% mannitol;
(c) 8-12 mM histidine; and
(d) 0.25-2% sucrose;
wherein the pH of the composition is 5.0-6.0.

24. The composition of claim 15 comprising:
(a) 1-3 mg/mL ularitide;
(b) 4-8% glycine; and
(c) 8-12 mM histidine;
wherein the pH of the composition is 5.0-6.0.

25. The composition of claim 17 comprising:
(a) 1-3 mg/mL ularitide;
(b) 4-6% glycine;
(c) 8-12 mM histidine; and
(d) 0.25-2% sucrose,
wherein the pH of the composition is 5.0-6.0.
